(19) United States
(12) Patent Application Publication
(10) Pub. No.: US 2019/0080799 A1
(43) Pub. Date: Mar. 14, 2019

(54) IDENTIFYING AND TARGETING PERSONALITY TYPES AND BEHAVIORS

(71) Applicant: Sony Interactive Entertainment LLC, San Mateo, CA (US)

(72) Inventor: Albhy Galuten, Santa Monica, CA (US)

(21) Appl. No.: 16/124,735

(22) Filed: Sep. 7, 2018

Related U.S. Application Data
(60) Provisional application No. 62/556,231, filed on Sep. 8, 2017.

(51) Int. Cl. G16H 50/20; G06Q 30/02; G06N 5/04; G06N 99/00

(52) U.S. Cl. CPC G16H 50/20; G06N 99/005; G06N 5/04; G06Q 30/0269

(57) ABSTRACT

The present invention is a method and system for determining the behavioral type of individuals or groups of individuals based on the psychological models of Jung, Keirsey, Myers and Briggs and using that information to make informed suggestions, optimizations and advertising targeting about those individuals. Moreover, this system may use various types of artificial intelligence to create arbitrarily granular detail of psychological typing so as to be able to apply it broadly. This modeling may be continuously updated and refined as the system learns about the degree of success of each of its recommendations.

### CROSS-REFERENCE TO RELATED APPLICATIONS

The present application claims the priority benefit of U.S. provisional patent application No. 62/556,231 filed Sep. 8, 2017, the disclosure of which is incorporated by reference herein.

### BACKGROUND OF THE INVENTION

### 1. Field of the Invention

The present invention relates to automated psychological analyses of consumer behavior, and delivering targeted information based on the same.

### 2. Description of the Related Art

Since Carl Jung first came up with Archetypes (beginning around 1919), psychologists have been using that kind of methodology to understand human behavior. In fact, this kind of analysis dates back to the 5th century BC. The terms sanguine, choleric, melancholic and phlegmatic were coined by the Greek physician Aelius Galenus to describe the effect of the humours on personality. A few years after Jung's description of archetypes, Katharine Cook Briggs and her daughter Isabel Briggs Myers, developed a mechanism for testing people to determine their type. Tests based on this Myers-Briggs Type Indicator (MBTI), were first published in 1943 and are still in use.

Such personality assessment tests are traditionally given in the form of questionnaires, however, which are prone to human biases and defensive mechanisms that may distort the results. Moreover, such traditional questionnaires generally result in categorizing the individual into strict dichotomies, which does not reflect much nuance, granularity, or conditions under which the individual may be categorized. For example, an individual may exhibit strong extroversion tendencies in professional situations, while exhibiting a different level or even introversion in personal situations. Moreover, different personality types as identified based on such questionnaires may nevertheless manifest in different behaviors.

Artificial Intelligence (AI including Machine Learning, Deep Learning, Expert Systems, Neural Networks, etc.) is getting more powerful daily. As these AI systems have access to more and more data, their capabilities are increasing exponentially.

As such, there is therefore a need for improved systems and methods of identifying and targeting personality types and behaviors.

### SUMMARY OF THE CLAIMED INVENTION

Broadly speaking, present invention leverages tendencies of different types of people to predict their behavior and enhance recommendations for them. The present invention uses Artificial Intelligence, Deep Learning, Machine Learning, Neural Networks, Expert Systems, and other software approaches to assist determinations of consumer behavior and psychological analysis.

In one example embodiment, the invention provides methods and system for analyzing the behavior of individuals and/or groups, and using that behavior to determine their Myers-Briggs type or similarly to determine their type along a Myers-Briggs-like typing continuum. Note that even though MBTI breaks people into 16 different types, there is actually a continuum of tendencies between the elements of the types, and AI systems are capable of an almost infinite degree of granularity. For example, this typing mechanism can then be used to determine individual and group proclivities and then be used to make recommendations, suggestions, optimizations and select target advertising to individuals based on how they map against those groups. Additionally, the systems can learn from behaviors based on the responses to the recommendations creating a feedback loop which can be used to more accurately define the typing mechanisms.

The present invention therefore provides for systems and methods of identifying and targeting personality types and behaviors.

### BRIEF DESCRIPTION OF THE DRAWINGS

The above and still further objects, features and advantages of the present invention may become apparent upon consideration of the following detailed description of some specific embodiments thereof, especially when taken in conjunction with the accompanying drawings wherein like reference numerals in the various figures are utilized to designate like components, and wherein:

FIG. 1 is a high-level view of the Component Architecture.

FIG. 2 is a view of the Elements of Myers-Briggs Type Indicators.

FIG. 3 is a view of the four temperaments of Myers-Briggs Type Indicators.

FIG. 4 is a view of the four temperaments compared on the axes of Abstract/Concrete and Cooperative/Utilitarian

FIG. 5 is a view of factors associated with Abstract/Concrete.

FIG. 6 is a view of the four Myers-Briggs temperaments seen through the lens of Tactics, Logistics, Strategy and Diplomacy.

FIG. 7 is a view of the path from MBTI to MBTI+ to MBTI++.

FIG. 8 is a view of the relationship between Collected and Observed data.

FIG. 9 is a view of an MBTI+ Array.

FIG. 10 is a view of how careers might be broken down through the lens of MBTI+.

FIG. 11 is a view of how behaviors and proclivities might be broken down through the lens of MBTI+.

FIG. 12 is a view of Mapping Data to Customization to Type.

FIG. 13 is a view of MBTI++ is enhanced using an analysis of actual usage

FIG. 14 is a view of predicted behavioral tendencies by personality type.

FIG. 15 is a block diagram of an exemplary electronic entertainment system.

### DETAILED DESCRIPTION

### Overall Component Architecture

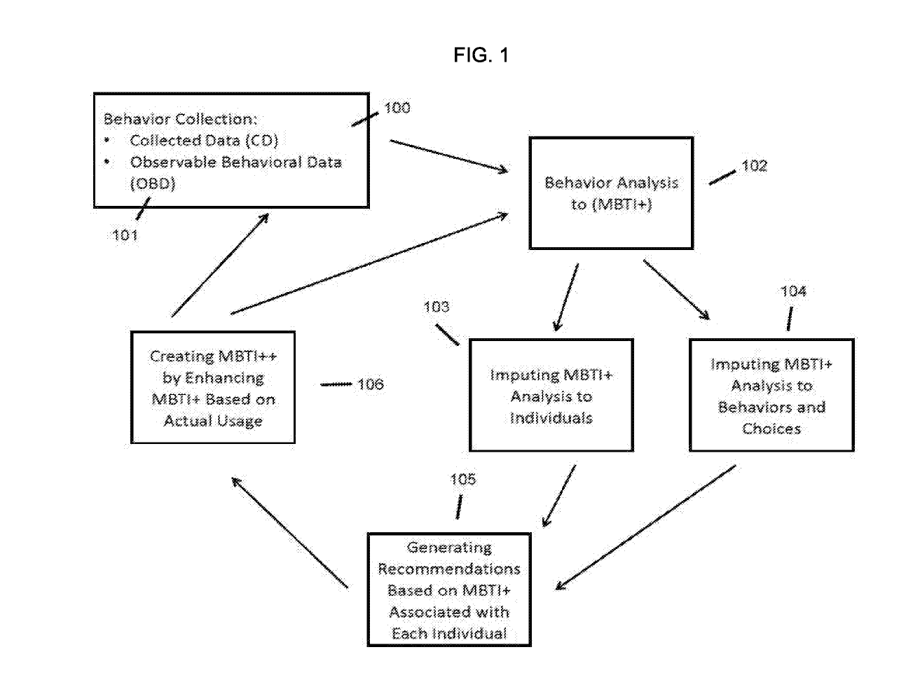

This method and system is broken into a number of components that when taken together, may represent an embodiment of the complete system as shown in FIG. 1 (but described in more detail below). The architectural components may be broken down as follows. First, there is the collection of the behavior from various sources, including but not limited to individual tests taken, additional analysis by professionals, and mapping against known tendencies of various groups of people, collectively referred to herein as Collected Data or CD (100). Less in the beginning but more and more as the system matures, such collected data on how people behave may include data collected regarding either real life (e.g., tracking their movements using their mobile phones), behavior on the internet, and use of social media (101). This set of collected data may be referred to as Observable Behavioral Data (OBD) and as the system matures, may come to play a larger and larger role (e.g., assigned greater weight) in the system.

These data are mapped against known Myers-Briggs Type data but in a more granular structure, so that there are not only the 16 Myers-Briggs types but a granular continuum of type elements that can be structured in arrays for use by computer systems. Such mapped data against Myers-Briggs Type may be referred to as MBTI+ (102). Then, once there is a large set of data regarding behaviors and tendencies, the Myers-Briggs Typing tendencies (or MBTI+ Data) can be associated with individuals and groups (103), including individuals and groups about whom no MB Type data is currently known. In parallel, expert reports may be used to predict and refine which MB Types have which lifestyle and usage tendencies (shopping, media consumption, etc.). The expected usage proclivities may then be mapped to the appropriate user base (104) and used to make recommendations and lifestyle (e.g. browsing, game playing, etc.) enhancements (105). Then using the analysis of the success of various recommendation and lifestyle choices (e.g., user feedback regarding proposed media options, further iterations are implemented to improve the accuracy of the model (106). This iteration process may be continuous, ever enhancing the ability to recommend and suggest media options that meet with a user's approval. What follows are the individual elements of the component architecture.

Granular MBTI

An explanation of Myers-Briggs Typing provides a foundation to the proposed enhancements and the other elements disclosed herein. MBTI or Myers-Briggs Type Indicators are based on 4 pairs of tendencies:

:Introversion (I)⋅Extroversion (E)

:Sensing (S) ⋅iNtuition (N)

:Thinking (T) ⋅Feeling (F)

:Judging (J) ⋅Perceiving (P)

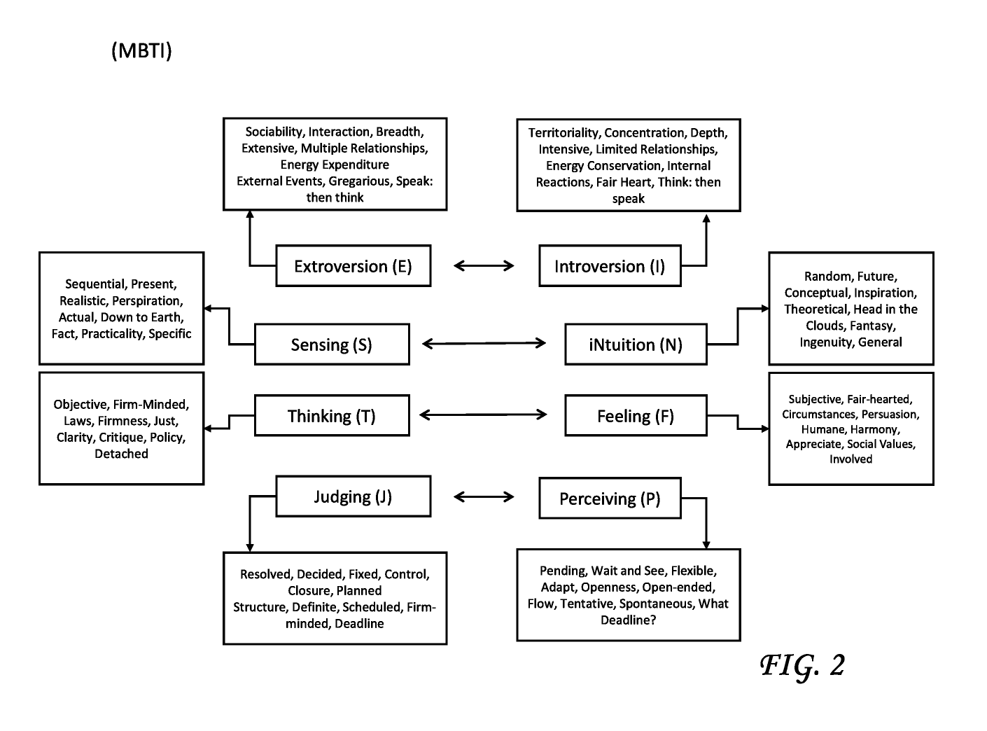

In different combination, the four tendencies create 16 possible types: ISTJ, ISFJ, INFJ, INTJ, ISTP, ISFP, INFP, INTP, ESTP, ESFP, ENFP, ENTP, ESTJ, ESFJ, ENFJ and ENTJ. Myers-Briggs Typing associates different characteristics with different types. As can be seen in FIG. 2, Extraversion is associated with Sociability, Interaction, Breadth, Extensive, Multiple Relationships, Energy Expenditure, External Events, Gregarious, and Speak: then think tendencies and behaviors. By contrast, Introversion is associated with Territoriality, Concentration, Depth, Intensive, Limited Relationships, Energy Conservation, Internal Reactions, Fair Heart and Think: then speak tendencies and behaviors.

Sensing is associated with Sequential, Present, Realistic, Perspiration, Actual, Down-to-Earth, Fact, Practicality and Specific tendencies and behaviors. In counterpoint, Intuition is associated with Random, Future, Conceptual, Inspiration, Theoretical, Head-in-the Clouds, Fantasy, Ingenuity, and General tendencies and behaviors.

Thinking is associated with Objective, Firm-Minded, Laws, Firmness, Just, Clarity, Critique, Policy and Detached tendencies and behaviors. Meanwhile, Feeling is associated with Subjective, Fair-hearted, Circumstances, Persuasion, Humane, Harmony, Appreciate, Social Values and Involved tendencies and behaviors.

Judging is associated with Resolved, Decided, Fixed, Control, Closure, Planned Structure, Definite, Scheduled, Firm-minded and Deadline tendencies and behaviors. Finally, Perceiving is associated with Pending, Wait and See, Flexible, Adapt, Openness, Open-ended, Flow, Tentative, Spontaneous, What Deadline? tendencies and behaviors.

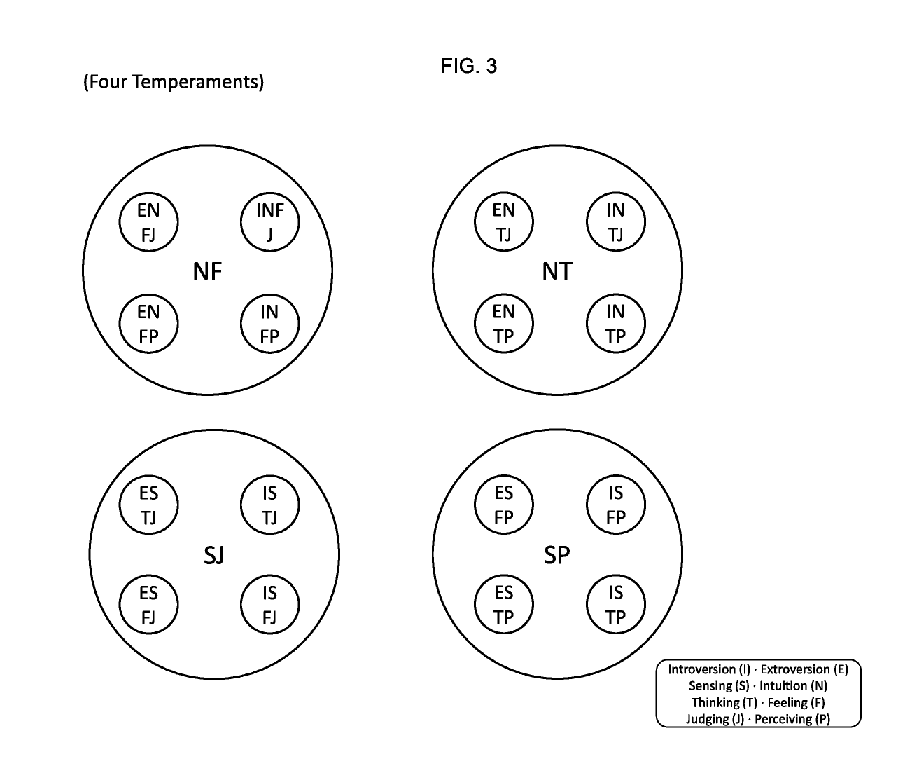

According to Myers-Briggs, as can be seen in FIG. 3, these 16 types may be grouped into 4 Temperaments: NF, NT, SJ and SP. Such groupings are not necessarily a less granular view, but rather a methodology for grouping personalities and traits.

As can be seen from the above, there is much nuance to each type, and with modern computational techniques, personality and behavioral analysis can be expanded beyond 16 types. On the JP axis, for example, there is no reason to limit it to either J or P. People (or organizations) can be anywhere on the scale from J to P, so one person might be 90% J and 10% P and another the opposite: 90% P and 10% J. People could fall anywhere on the scale including 50% J and 50% P, which may make them neither or both J or P in the traditional MBTI model. This same logic can, of course, be applied to all the axes: I-E, S-N, and T-F, as well as J-P. The enhanced analysis of people's MBTI involves applying a granular scale, so that each tendency in a pair is represented for each individual (e.g., by a percentage of each).

There is, however, much more historical data (which was based on traditional MBTI with typically only 16 variants) than such granular data. As such, the specific percentages may be set as unknowns by default. Analyses by psychologists of the specific answers to the standardized questions may initially be used as a starting point for the default percentage. For example, a panel of psychologists may predict that a specific set of responses to specified questions result in the respondents being grouped as 75% I and 25% E; then for all historical MBTI scores matching those responses, such predicted breakdowns may be used as the default percentages. However, more relevant and current data is accumulated over time, the percentages may be adjusted accordingly. In some cases, the percentages may only apply to those subjects for whom there is currently no deeper degree of granularity for the data. For subjects for which more detailed answers are known or for whom there is other more detailed data, a more nuanced score may be derived. Also, going forward as even more nuanced data is gathered based on more data points (as discussed further herein), the relevant weight of the answers to each question and to other behaviors and questions may be adjusted.

Other Axes

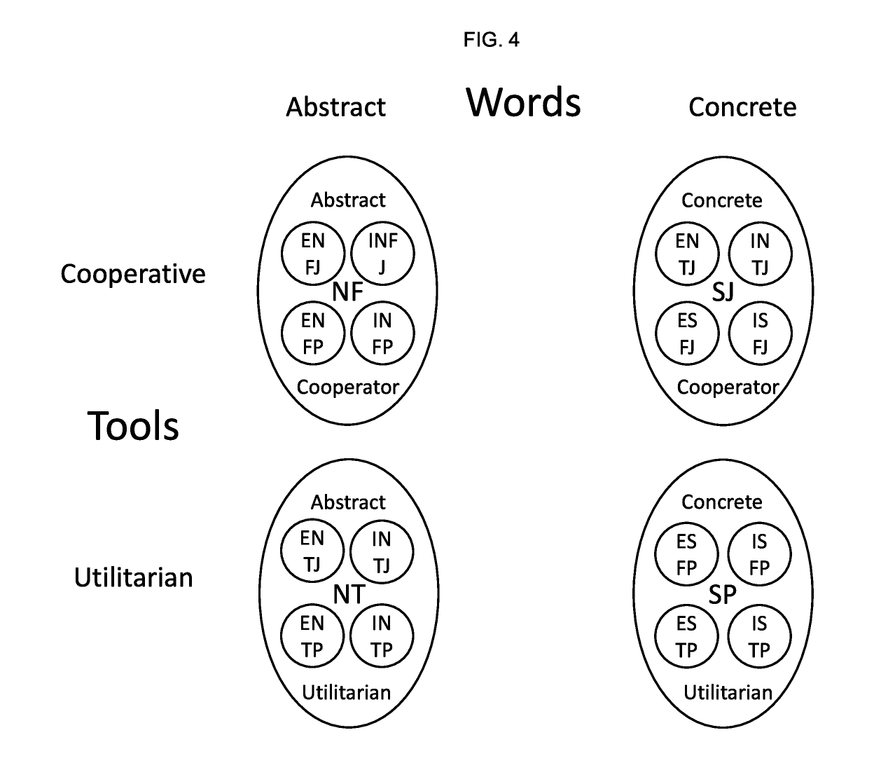

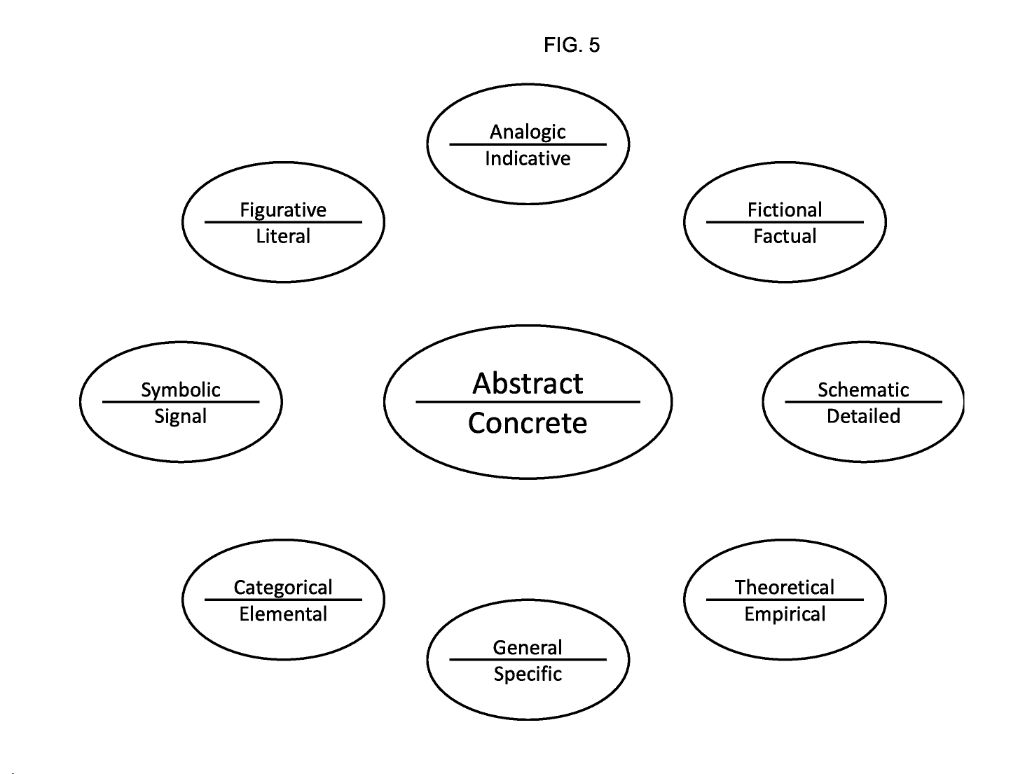

Though Myers-Briggs breaks the 16 types into 4 primary pairs, in other analysis done by David Keirsey, these same types can be described along the two axes of Words being Abstract/Concrete and Tools being Cooperative/Utilitarian. As can be seen in FIG. 4: NFs are Abstract and Cooperative, SJs are Concrete and Cooperative, NTs are Abstract and Utilitarian and SPs are Concrete and Utilitarian. As can be seen in FIG. 5, some of the terms used to describe the sides of Abstract/Concrete are: Analogic/Indicative, Fictional/Factual, Schematic/Detailed, Theoretical/Empirical, General/Specific, Categorical/Elemental, Symbolic/Signal and Figurative/Literal. As one might imagine, databases of synonyms and antonyms can be used to ascribe other behaviors to various descriptive levels on the Analog/Concrete Axis as can be done on the Abstract/Concrete axis. Because AI systems can learn to ascribe levels of “matching-ness” to large sets of lexical data, these lists can be enhanced and weighed for relevance to different type matchings.

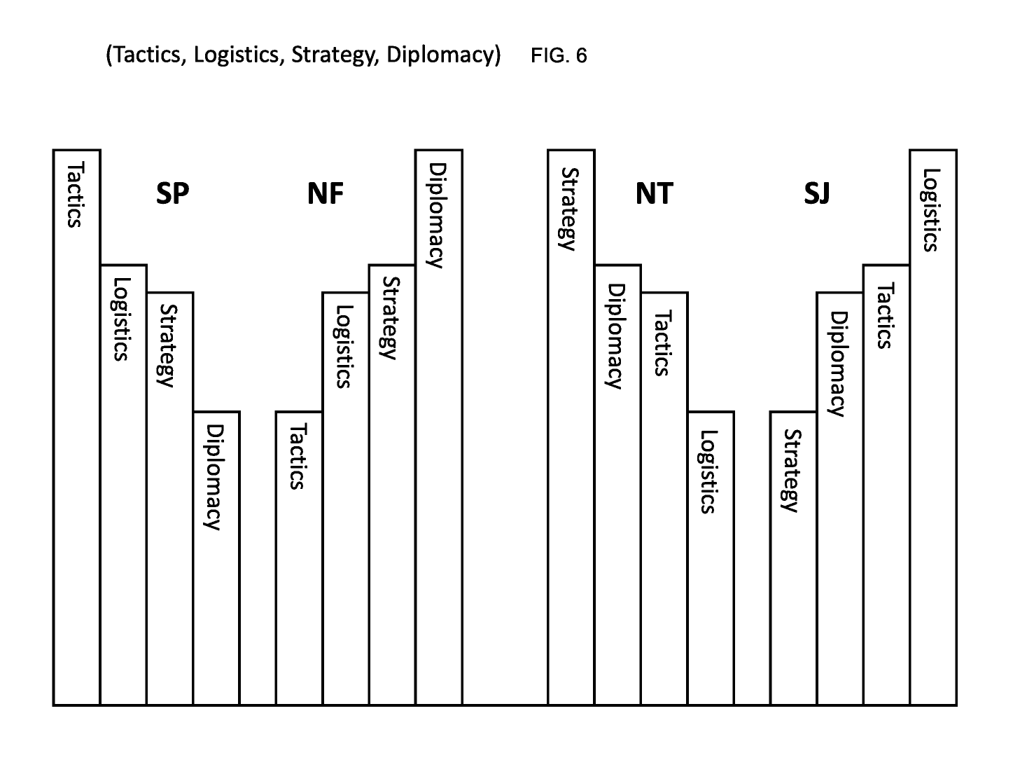

Keirsey also breaks the types down along the lines of Tactics, Logistics, Strategy and Diplomacy and associates relative weights of those characteristics to the four types as can be seen in FIG. 6. Keirsey's initial weightings, again, represent a starting point but AI learning systems may be able to define degrees of Tactics, Logistics, Strategy and Diplomacy as associated to each individual or group with greater specificity.

All of the above work by Myers-Briggs and Keirsey lays the foundation for computer analysis. Other psychologists, psychiatrists, sociologists, anthropologists, human resource experts, educators and philosophers have contributed to the field. These experts may be used to seed the initial typing data but, ultimately, AI may generate a complete learning system to use and enhance this data for the purpose of enhancing recommendations and optimizing user experience. Based on the success of each recommendation and the follow-on observations of individual behaviors, the system may continue to learn and enhance its capabilities in a virtuous learning cycle.

The Path to MBTI++

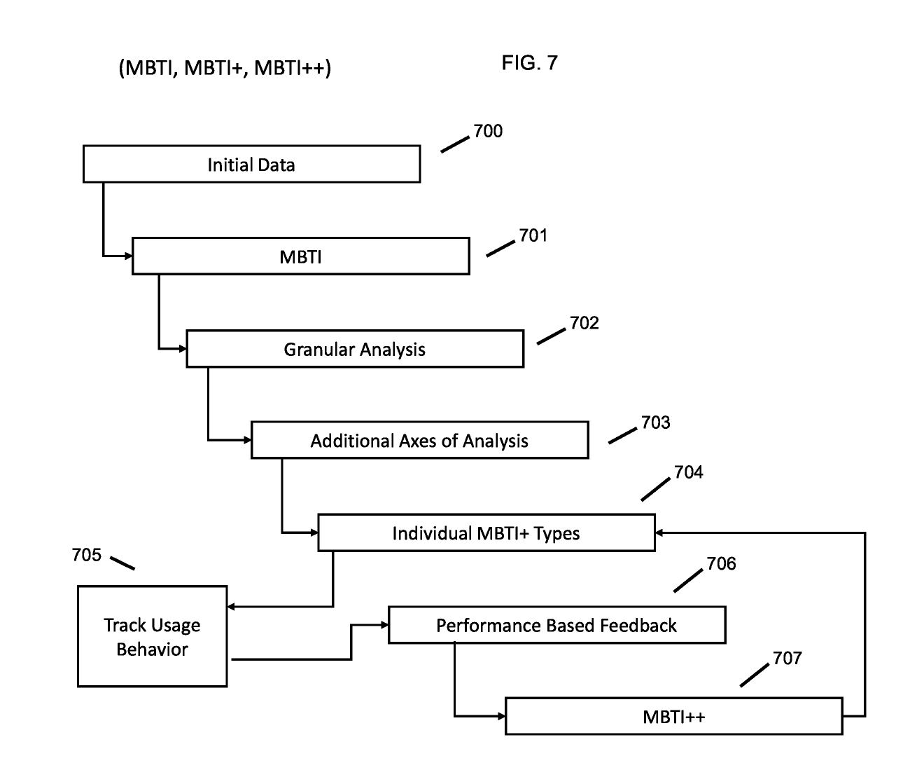

To look at the overall architecture from a different perspective, a system may begin with MB Type data and enhance it until there is a more granular and more robust set of type data. As can be seen in FIG. 7, the system may begin with Myers-Briggs Typing as originally defined but enhanced and quantified. Starting from such initial data (700), that data may be mapped onto MB Types (701). Next may come a Granular Analysis (702) phase, where the degrees of each type are quantified. After that, Additional Axes of Analysis (703) may be added, such as Tactics, Logistics, Strategy, and Diplomacy, as well as one multi-access view and Abstract/Concrete and Cooperative/Utilitarian as an additional pair of axes. These enhancements to MBTI create a continuum of types, which may be referred to herein as MBTI+(704).

In the next phase (705), feedback may be received on how these people and groups act in real life. This is fed into a Performance Based Feedback Module (706), which uses these enhanced observations (data) and these analysis metrics to arrive at MBTI++ (707). This process is iterative and continuous, constantly refining the model with MBTI++ (707) data feeding back into the data of the individual MBTI+ users and groups (704). These enhanced types may not only be arrays of relative degrees of I-E, S-N, T-F and J-P, but may be relative with regard to the relevance and weighting to each usage behavior creating a predictive model that can be used to optimize user experience and recommendation.

Collection of Behavior

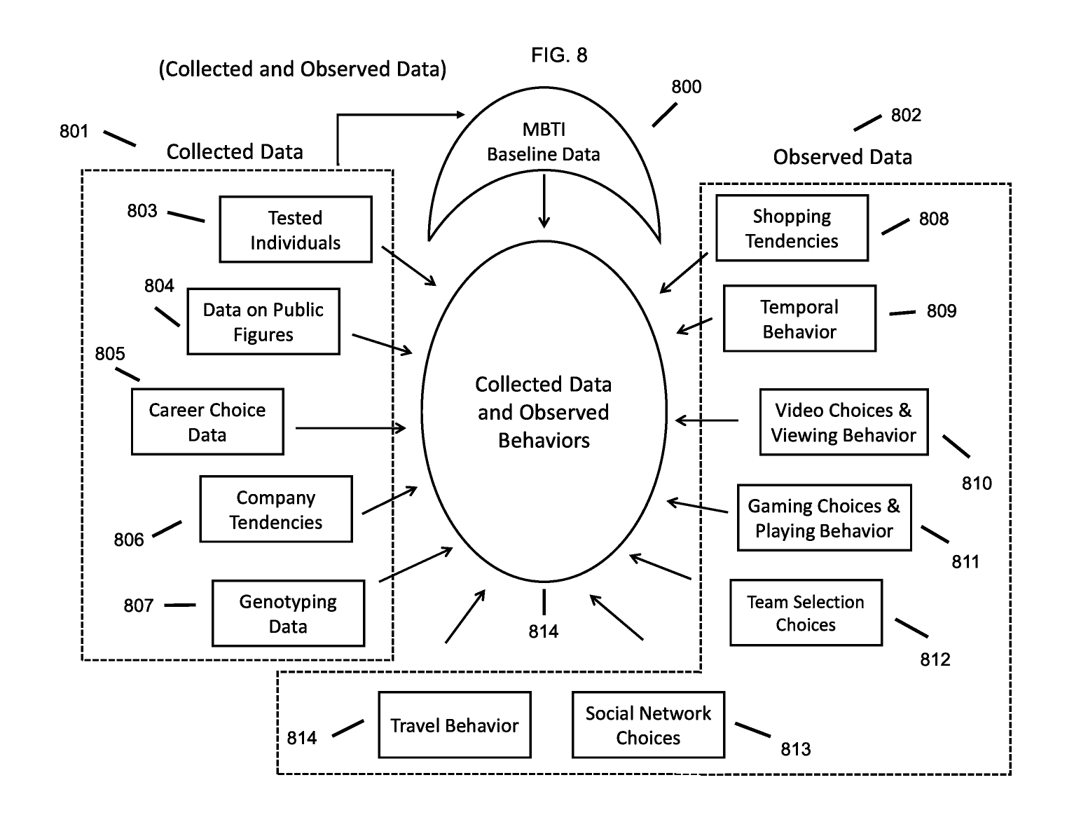

Before one can do an analysis based on how closely an individual or group shares the traits of an MB Type (MBTI), one may determine what traits are associated with each type. This can be done in a number of ways. As can be seen in FIG. 8, many different sources may be used to create a set of MBTI Baseline Data (800). Generally, the data is broken into two different types: Collected Data (801) and Observed Data (802). This data can then be used as a yardstick to measure which MB Type an individual or group should be most closely associated with. This is only the first step, and it should be noted that once the system begins to learn, the AI of the system may begin to create many more types than the 16 MB Types. Some embodiments, however, may start with an Expert System. The first thing needed for an Expert System is a knowledge base. Such a knowledge base may be seeded by some combination of the following elements as well as other elements that can add to the knowledge base.

The following is an initial list, one practiced in the art of psychological analysis can find and use many more:

Volunteers who have taken an MBTI test and agree to or have already filled out a questionnaire or in some way allow a system to map their MB Type against other factors (803).

Data about well-known people and their believed typing based on the analysis of psychologists or other experts in the field (804).

Data and analysis about career choices and tendencies toward Type (805). There is a wealth of available data regarding which MB Types are best suited for particular careers. Starting from that point, potential MB Types may be ascribed to each career and using expert analysis and eventually more accurate retrospective data to determine the amount to weight each career choice. For example, career might be extremely indicative of a particular MB Type, but another career might be only loosely associated or not associated at all. Such data may include the following:

Data and analysis about company tendencies toward Type (806).

Data and analysis using genotyping (genomes of individuals who have agreed to submit their genome as part of a psychological analysis) and its relationship to Type (807).

Data and analysis about shopping tendencies and their relationship to Type (808).

Data and analysis about temporal behavior and its relationship to Type (809).

Data and analysis about video choices and viewing behavior and its relationship to Type (810).

Data and analysis about video game choices (811) and playing behavior (812) and their relationship to Type.

Data and analysis about social affiliations and their relationship to type (813) including selection of team mates in online gaming and the success of various type combinations on team success.

Data and analysis about travel behavior and its relationship to type (814) including everything from regularity at a local coffee shop to countries visited and time of stay and time of year and age of person when traveling.

As noted above, association with a Type is nuanced and not binary. The system may try to, when the information is available, weight the proclivity. For example, a person, group, company, career group, behavioral tendency, etc., may not be either I (Introvert) or E (Extravert), but rather a percentage of one vs the other. For example, one person or behavior might be 80% E and 20% I, while another might be 55% E and 45% I. As demonstrated herein, the value of each tendency may not only be divided based on its percentage of different components, but may also be weighted based on the overall value of that component to the whole.

Initial Behavior Analysis

First, an array may be generated in which to store all the basic MBTI+ data, e.g., regarding the four pairs of tendencies: Introversion (I)/Extroversion (E), Sensing (S)/iNtuition (N), Thinking (T)/Feeling (F) and Judging (J)/Perceiving (P) or I/E, S/N, T/F and J/P. Each pair make up 100%, so the array may simply include the first number of each pair. For example, if the value of I is x %, then the value of E is 100-x %. For the array mapping below, it can be seen that only four numbers are needed.

| I | E | S | N | T | F | J | P |
|---|---|---|---|---|---|---|---|
| I % | 100% - I % | S % | 100% - S % | T % | 100% - T % | J % | 100% - J % |

| I | E | S | N | T | F | J | P |
|---|---|---|---|---|---|---|---|
| 85% | 15% | 17% | 83% | 43% | 57% | 62% | 38% |

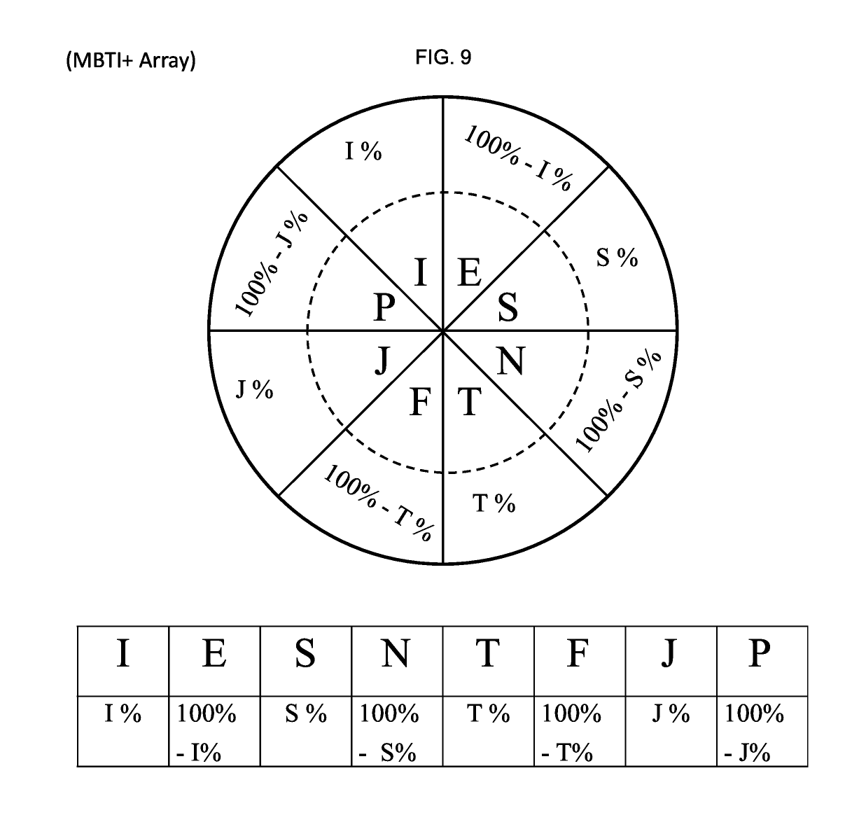

Two different graphical representation of typing to arrays can be seen in FIG. 9.

This is one weighting for one person or group based on one set of observations and of collected data. An individual person or group might have multiple arrays (or perhaps, in development one larger multidimensional array). For example, one proclivity might apply to assembling teams for an online video game battle, and another might apply better to shopping suggestions, and still another to video consumption.

In doing the behavior analysis, the initial analysis may be weighted for veracity based on presumed (e.g., default) and/or historically analyzed weightings. So for example, if a board of psychologists believe that architects are most commonly ENTJ and may, on average be weighted as: I: 40%, E: 60%; S: 20%, N: 80%; T: 75%, F: 25%; J: 55%, P: 45%, (or as an array: [40, 60, 75, 55]). Such weighting may be an initial starting place for architects. One can do weightings for all other professions and behaviors. Another example might be people who check out in an online store as quickly as possible, while others like to double check their choices. For those who like to double check their choices, a “double-check screen” can be generated as part of the check-out process. This may be more satisfying for them and may mitigate some failure to complete check out. This is but one small example. Others may be described further in this method and process but further than that, the AI systems may be able to tabulate other behavior optimizations (based on observing behavior and results) that could not be predicted by human analysis.

Using a similar “Expert-System-Like” mechanism, a similar initial analysis may be applied to: Social Network Choices, Frequency of Checking, Browsing Behavior, Game Choices, Game Play Behavior, Calendar Behavior, Genetic Predisposition, Video Viewing Behavior, Social Behavior, Shopping Behavior, Location Based Behavior and Temporal Behavior. From these starting positions, the AI may develop other axis and granularities.

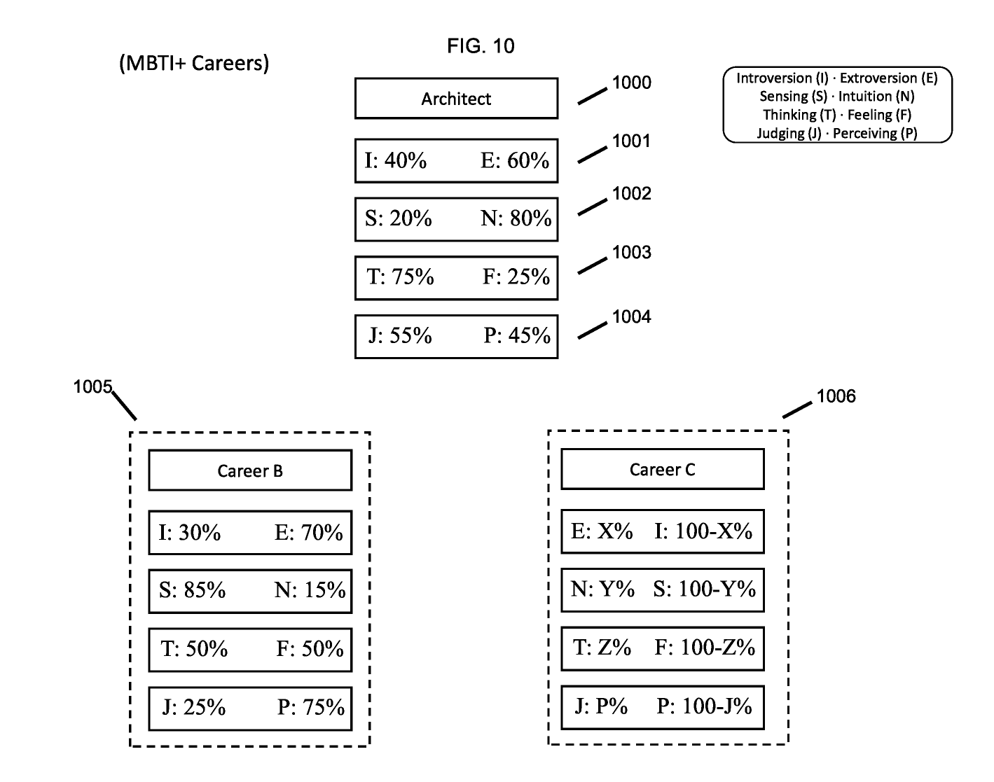

Looking at FIG. 10, you can see some examples of potential breakdowns by career. As more data is collected on individuals, an AI can determine how to weight every axis, and this can be done for every career and every sub-career. For example, the refined analysis might find that architects are relatively true to their ideal type while pediatricians have extreme variability with regard to type. So when deriving a person's type, the analysis might find that being very close to the most common “Architect Type” has a higher likelihood of that person being an architect (e.g., 15%), while a person who is very close to the most common “Pediatrician Type” has only a 3% chance of actually being a pediatrician. In this phase of the data construction and usage model, such career data may be used to guess at type—not to be confused with the reverse—which may be done later—which is to guess at a person's expected choices, preferences or proclivities based on their type.

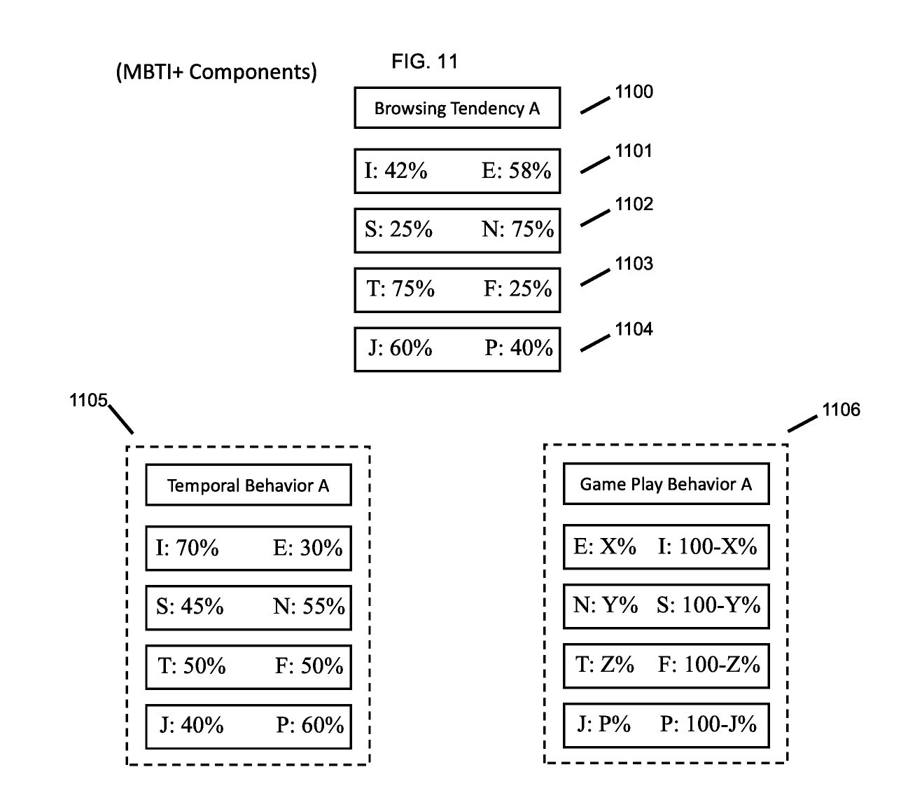

FIG. 11 shows three possible breakdowns for behavioral tendencies.

Mapping Behavior Analysis to MBTI and MBTI+

The ultimate goal of this system and method is to use data that has been collected passively by observing user behavior. That behavior is then mapped against MBTI+ quantifications, which can later be used as a basis for recommendations and usage experience enhancements.

The data may be grouped into two groups of data: Collected Data (CD) and Observable Behavioral Data (OBD). Separating out the list from above (as seen in FIG. 8):

The Collected Data may include:

Volunteers who have taken an MBTI test and agree to or have already filled out a questionnaire or in some way allow a system to map their MB Type against other factors.

Data about well-known people and their believed typing based on the analysis of psychologists or other experts in the field.

Data and analysis about career choices and tendencies toward Type.

Data and analysis about company tendencies toward Type.

Data and analysis using genotyping (genomes of individuals who have agreed to submit their genome as part of a psychological analysis) and its relationship to Type.

The OBD may include:

Data and analysis about shopping tendencies and its relationship to Type.

Data and analysis about temporal behavior and its relationship to Type.

Data and analysis about video choices and viewing behavior and its relationship to Type.

Data and analysis about video game choices and playing behavior and its relationship to Type.

Data and analysis about social affiliations (friends on social networks, etc.) and their relationship to type including selection of team mates in online gaming and the success of various type combinations on team success.

Data and analysis about travel behavior from regularity at a local coffee shop to countries visited and time of stay and time of year and age of person when traveling.

Taking all of the above data into consideration, some starting values for each proclivity may be developed. Based on usage and historical analysis, these values may change. However, a starting point (Expert System) may need to be identified. As discussed further herein, each of the above data sets might be associated with behaviors within those sets to MBTI+ numbers. Although these specific data sets are discussed herein, one practiced in the ordinary psychological arts could create many more possible groupings.

Starting with CD (Collected Data) sets one at a time:

Tested Volunteers: there are two groups of volunteers. First, there are those who have taken the test but given no additional data. This group of data is useful in a general sense. It can indicate the percentages of different groups that fall into different categories and in cases where there is specific test results (not just the numbers), the specific answers to individual questions may be used to weight the tendency toward one side or the other of a pair (e.g. I-E, T-F, etc.). There has already been some analysis of how to weight specific answers, and more can be done. The second group of volunteers may include volunteers who have agreed to provide additional data (e.g., profession, schooling, sleep patterns, preferred shopping methods, television and game playing preferences). Although sometimes what people say they do and what they actually do are different things, nonetheless this set of collected data may be invaluable for making initial predictions about MBTI+ and behavioral choices. Note that the most valuable contingent of subjects may be those who a) take the MBTI test, b) answer a number of additional behavioral questions, and c) allow for their respective behaviors online to be tracked. This tracking could be an opt-in on a social network or a broad shopping site or a gaming network or a video service.

Data about well-known people and their believed typing based on the analysis of psychologists or other experts in the field. There have been many analyses of famous people based on their known psychological tendencies where experts have ascribed MBTI data to those famous people. There is frequently much other data known about famous people and that can be used to expand the depth of knowledge about their Type just as answering the additional questions above can help inform and create corollary information that can be associated with type. Historically, MBTI data was designed to help people deal with their psychological universe only. The present mapping, however, accounts for their respective functional universe, and the mapping of functional data (e.g., did Lincoln like to shop, does Barbara Streisand collect things, etc.) may therefore yield a different kind of information about various MBTI types.

Data and analysis about career choices and tendencies toward Type. Much work has been done in this area, and there is a wealth of data to draw on. Experts in Human Resources and psychology can pore through the data and enter in a predetermined format that can be consumed by the system, including one or more MBTI+ sets of imputed data about each profession (there could be multiple types associated with each profession) and associating it with a relevance score. One profession might be highly correlated with certain types and other profession much less so. So for each profession, there may be one of more MBTI+ arrays and there may also be a relevance score to be used for weighting the importance of this typing to the profession and the likelihood of these workers to be a member of that MBTI+ array.

Data and analysis about company tendencies toward Type. Just as some careers tend to be more of one type than another, some companies have the same tendencies. Again, a panel of experts can predict an MBTI+ array to associate with a company and a relevance score for that association. Over time as the contingent of subjects grows and more data is learned about those subjects, the AI may update its assumption, thereby regularly increasing its accuracy.

Data and analysis using genotyping (genomes of individuals who have agreed to submit their genome as part of a psychological analysis) and its relationship to Type. It does appear that MBTI typing is very deeply ingrained, and many of the behaviors are observable even in infants. One might reasonably expect that there may be a genetic component to the different types. As more genome data becomes available and can be mapped against the MBTI+ typing, tendencies may appear, and genotyping can then be used to predict MBTI+ types—again with a relevance score associated.

Adding the OBD (Observable Behavioral Data) sets: these data sets are most valuable when provided by people who have taken MBTI tests and also agreed to be tracked to gather enhanced data. However, because there may be other groups of people who have been typed, the data on those people who are only tracked more passively may increase in value as time goes on. For example, a contingent of people may be observed to be very meticulous whenever they check out using their online shopping cart, and it might be presumed that they are more S (Sensing) than P (Perceiving). This assumption can be compared with the behavior of known MBTI types and may turn out to be accurate with a 56% percent relevance score. As more touch points are added, the model may refine its own accuracy in a positive feedback loop. So for example, the checkout process in the store may be optimized for people of a specified type. Then, double blind comparative analysis, may determine how much of an uptick in successful shopping results from that optimization. Then, as different shopping experiences may be offered to different people based on how different people gravitate toward (or perform better using) certain check-out approaches, MBTI+ data may be associated with those people who gravitate toward different check-out processes.

As a corollary, consumer preference setting data may be used as an input to typing data. For example, people who turn off more notifications on their phones that then others may be more likely to be I (Introverted) as opposed to E (Extroverted).

Many other behavioral choices can be used by psychologists with expertise in the field and the choices given above should not limit the possible choices that one practiced in the appropriate fields may choose.

Data and analysis about shopping tendencies and its relationship to Type. As discussed herein, such shopping may include the check-out process and its possible association with type, but there are many other components to shopping that can indicate type. Some parameters (again, more may be suggested by experts in the psychology of shopping and the lists and associations of components may be pruned and augmented based on retrospective behavioral data) for shopping tendencies might be: a) how often does someone shop, b) for how long, c) what kinds of things do they shop for, d) how much do they use wish lists, e) do they leave things in their cart and come back to them later or do they always empty the cart when they have finished a shopping session . . . etc.

Data and analysis about temporal behavior and its relationship to Type. Temporal behavior is a very rich and varied domain: a) how long is their commute (one might say that is not a choice but some people may avoid a long commute at all costs and others don't care so much)? b) How much sleep do they get? Do they wake in the middle of the night, etc.? c) How much do they vary their regular routes (to work, home, the gym, etc.)? d) How early do they get to the airport for a flight? e) Are they typically early or late to meetings? By how much? f) How regular are their meal times? Trained psychologists can use many other metrics in the temporal domain to make associations to type.

Data and analysis about video choices and viewing behavior and its relationship to Type. There have been studies done where specific movies are suggested to people based on their MBTI score. Again, some reasonable choices may be made based on the best guesses of experts, but by tracking the success of the recommendations, the accuracy may blossom when there is sufficient data. For example, an individual may be typed as above:

E: 60%, I: 40%; N: 80%, S: 20%; T: 75%, F: 25%; J: 55%, P: 45% or as an array: 60, 80, 75, 55. Data regarding what movies they like may be examined, and using Collaborative Filtering, those movies may be used as recommendation to others with similar types. Additional data may be indicate if they watch the movie all the way through, don't watch it at all, or watch it multiple times, etc., as indicators of success. Over time, data about which video choices proved to equate to what types may reveal themselves. Some types and some titles correlate very little, but other types or videos correlate very highly.

Data and analysis about video game choices and playing behavior and its relationship to Type. Here, there may be some similarity to the approach taken with video above, and typing and observable success may be used to predict what video games a type of player might like. However, with gaming choices there are much more nuanced behaviors to be tracked and analyzed. For example: how active or passive does a person play, for how long do they play, do they tend to play offensively or defensively, how do they respond to puzzles, to incentives, to rewards. How social are they in their gaming. Do they like to watch the intro or just get into it? What kind of teammates do they gravitate toward in online game play? Do they like to organize their trophies, spells, etc. or don't they care. What time of the day are they social as opposed to more social, etc., many more parameters can be considered. All of these parameters are effected by MBTI++ typing and knowing someone's typing parameters can help to optimize their gaming experience.

As an example, if a certain MBTI++ type likes to gather his or her team for a first person shooter game before s/he begins playing and another MBTI++ type likes to just start playing and find teammates after s/he is familiar with the game, this can give the game designer a mechanism for directing game play and set up for each individual based on their MBTI++ type.

Data and analysis about social affiliations (friends on social networks, etc.) and their relationship to type. Clearly MBTI++ typing is indicative of many parameters and so one's behavior on social networks can be very indicative of type. There is a likelihood that people may gravitate more toward people of similar types. Behavior online is indicative of type. Certainly an elementary AI can, monitoring behavior online, easily guess the Introvert/Extrovert scale. But other scales are also very dependent on behavior. People who follow and participate in philosophy and policy discussions are more likely to be Perceiving as opposed to Sensing. Again, a proposed set of expected behaviors may be based on expert opinion regarding association with various type parameters, and the AI learns as more data points are correlated. This can be tested by making suggestion and gauging their uptake, but also by correlating these users against other parameters as collected above and of course, against those who have taken MBTI tests.

Data and analysis about travel behavior from regularity at a local coffee shop to countries visited and time of stay and time of year and age of person when traveling. In the same way as other behaviors, MBTI types may be imputed from people's behavior. People who have their coffee at Starbucks at the same time every day are probably more sensing than perceiving, as is true for people who research trips far in advance and with a lot of detail.

As a general principle, MBTI is based on people filling out surveys where they say how they typically react in various situations (as well as how they feel). With opt-in, data may be obtained regarding what people actually do. Actual behavior is often a much better indicator of people's personality than what they say they may do.

Imputing MBTI Analysis to Behaviors and Choices

It should be noted that every association made to an individual's type can also be made to the type of a group. So, for example if it is determined that soldiers tend to be a group of types and that most of those types prefer certain approaches to shopping, one could logically predict that designing the shopping experience for soldiers (e.g. at the Camp PX or online store) based on that typing knowledge could yield a better shopping experience and consequently more and better shopping. It might also be different for the wives of the soldiers as opposed to the men soldiers themselves. Note this is not a sexist determination as the granularity can be to any degree including sets of just one individual.

One analogy to help clarify the process is translation. MBTI++ may be used as a kind of Esperanto to translate from people to behaviors and from behaviors to people. At this point, people's behaviors may be converted into or common behavioral language, referred to herein as MBTI++. Later, MBTI++ analyses may be used to map recommendations and optimization onto the users.

Our next task is to map these Enhanced Types or MPTI+ arrays to individuals. Certain behaviors may be associated more or less strongly with different behaviors (and potentially genetic factors). Now with an ever evolving set of correlations between various behaviors and MBTI+ data, the process may be inverted to try to associate MBTI+ arrays to individuals. For example, it may be determined that 34% of people who behave in a certain way (say they double check their electronic shopping cart before they finalize the transaction) are believed to be of a certain temperament (e.g., SJ which includes ESTJ, ESFJ, ISTJ and ISFJ). What that means is that in analyzing the probability that an individual with that habitual behavior is an SJ, this particular behavior may be weighted at 34%. Alternatively, if a behavior was believed not to be related to just a temperament but was correlated to a 4 letter type (e.g., they wrote detailed reviews of products of a very tactical nature, referred to their experience a lot in the review, and checked them multiple times before posting, and that was correlated to ESTJ) at 27%, then that behavior may be weighted at 27%. Now, to tally these various weightings, the data can be added and the tendencies weighted as indicated by their percentages and from that determine a likely type. Additionally, the frequency of these actions (as a total in time and as a percentage of collected actions) may be given a quantity.

Let's suppose subject “A” has the following indicators based on all their observed behaviors:

| | | | FRE- | TEMPORAL |
|---|---|---|---|---|
| | | | QUENCY | FREQUENCY |
| | | % OF | OF | PER |
| BEHAVIOR | MBTI | CORRELATION | BEHAVIOR | YEAR |
| 1 | ESTJ | 23 | 85% | 79 |
| 2 | ESTJ | 34 | 34% | 945 |
| 3 | ISTJ | 17 | 79% | 23 |
| 4 | ESFP | 84 | 28% | 149 |
| 5 | ISFJ | 11 | 13% | 678 |
| 6 | ISTJ | 39 | 97% | 45 |
| 7 | ESTJ | 34 | 48% | 1157 |
| 8 | INTJ | 54 | 53% | 5678 |
| 9 | ESFP | 23 | 75% | 456 |
| 10 | ISFJ | 61 | 67% | 52 |
| 11 | ESTJ | 23 | 34% | 56 |
| 12 | ISTJ | 21 | 29% | 47 |
| 13 | ISTJ | 35 | 67% | 3458 |
| 14 | ENFJ | 46 | 76% | 456 |
| 15 | ISFJ | 12 | 65% | 543 |
| 16 | ESTJ | 32 | 48% | 745 |
| 17 | ESTJ | 31 | 68% | 4785 |
| 18 | INTJ | 40 | 47% | 1243 |
| 19 | ESFJ | 28 | 54% | 345 |
| 20 | ISFJ | 19 | 67% | 624 |

Similar to the way in which Expert Systems may be used as a starting point for behavioral analysis, experts may guide the weightings of the various parameters. Having said that, AI may be used to fine tune and even replace the initial assumptions based on the success of each assumption. Looking at the chart above, the Frequency of Behavior and Temporal Frequency per Year are hard numbers that were collected but the percentage of correlation is, initially, subjective based on expert opinion. This number may change based on learning from the collected data and comparing it with the results of the behavior analysis. For example, experts may believe that SJ (including ESTJ, ESFJ, ISTJ & ISFJ) type people are likely to double check their shopping cart more than once 75% of the time, but in practice with observation of behavior, the system may find that it is actually only 65% of the time. The model may then be adjusted. For example, the system may begin with 75% assumption and find that it actually breaks down as ESTJ-75%, ESFJ-45%, ISTJ-90% & ISFJ-78%. So knowing that the percentages may adjust over time, algorithms may be developed to impute, at least initially the percentage of correlation. Like other variables, this algorithm is expected to evolve based on usage and behavioral data. So the algorithm may include variables as follows: Let's call the % of Correlation ‘c,’ the Frequency of Behavior ‘f,’ the Temporal Frequency per Year ‘t’ and the MBTI Type ‘m’ (in the generalized formula—in actual computations they may be the four letter type, i.e. INTP=).

The algorithm may be seeded with an initial formula of m=tMt fMf cMc where t is temporal frequency for the day in question multiplied by Mt, the Temporal Multiplier (the likelihood that this behavior is more or less likely than average to occur on this particular day); f is the frequency as a percentage of how relevant this should be weighted which is multiplied by Mf, the importance of this behavior on the likelihood of it being dispositive and c, the percentage of correlation which can be multiplied by Mc, a multiplier used to weight the correlation as a it compares to the two frequencies. Having started here, the system may adjust the inputs for t, f, and c. t might equal T (the actual number of events per year) divided by 365 to get an average number per day and perhaps that average might be weighted (based on the use case) to weigh weekends and holidays less heavily than weekdays by a percentage to be determined or could be calculated independently based on information about the day the measurements are being taken. Though there are many different possible choices for all the variables, it may help to clarify this example a bit. To find out for the equation, it may be made of a number of Ts each, potentially, with a different value. There are: regular weekdays, regular Saturdays, regular Sundays, 3 day holiday Mondays (Presidents day, etc.), but adding a bit more granularity (remember data storage and processing resources both trend toward the infinite), additional options may further include Thanksgiving, Thanksgiving eve, day after Thanksgiving, cyber Monday, etc., etc. Based on the relative importance of temporality in a particular use case, there may be a weighting for temporality, such as Mt above. Introverts become even more introverted on the day after Thanksgiving. Let's suppose that Mt327 represents the value for the Friday after Thanksgiving and that Mt325 represents the value for the day before Thanksgiving. Now presuming that ESTJs are likely to act like ESTJs 75% of the time on the Wednesday before Thanksgiving but only 45% of the time on the day after Thanksgiving, the calculation for the likelihood of someone acting like an ESTJ may place the tMt value for the day before Thanksgiving at 75% and for the day after Thanksgiving at 45%. This may indicate (among other things) about targeting the following

| | |
|---|---|
| INTJ | 6.23 |
| INTP | 4.49 |
| INFJ | 5.53 |
| INFP | 3.08 |
| ISTJ | 19.01 |
| ISTP | 2.49 |
| ISFJ | 11.24 |
| ISFP | 1.06 |
| ENTJ | 3.24 |
| ENTP | 5.6 |
| ENFJ | 7.04 |
| ENFP | 4.21 |
| ESTJ | 19.55 |
| ESTP | 7.07 |
| ESFJ | 23.5 |
| ESFP | 5.1 |

ESTJ-like behavior, it is better to do that the day before Thanksgiving as opposed to the Day after Thanksgiving. Further, using the example chart above to factor in the frequency of behavior from the data may indicate that ESTJs may act in the proscribed manner 85% of the time. This particular behavior (e.g., double checking the shopping cart before executing) may be 35% correlated to ESTJ. Additionally, the percentages of correlation to other types (i.e. INTP, 13%, ISFJ 7%, etc.) may be generated. So if someone does this particular behavior on the day before Thanksgiving, it may be known that the behavior correlation score is 0.1955. This doesn't mean that there is a 19.55% likelihood that they are ESTJs but rather that is their score. Using other factors and similar analysis, the score for each of the 16 types may be determined.

For example, scores for all the 16 types may be as shown in the table. The average likelihood (using a simple mean) may be 8.03%, but the average of the SJs is 18.33. From this one could conclude that SJs are more than twice as likely to double check their shopping cart as the average user. The inverse may also be true, that is that people who double check their shopping cart are more than twice as likely to be SJs as the norm. As projections are made and compared with the actual results, the new projections may become, increasingly, more accurate.

The above is one example. When actually building a system, thousands of variables may be used and compared. Using the Observed Data example list from FIG. 8 (802): Shopping Tendencies, Temporal Behavior, Video Choices & Viewing Behavior, Gaming Choices & Playing Behavior, Team Selection Choices, Social Network Choices, Travel Behavior (e.g., looking at categories or groups of behaviors). The example above of shopping cart double checking is a subset of Shopping Tendencies. Some other shopping cart behaviors might be: does/doesn't let device remember password, uses multiple lists (e.g. saving for later) or just uses one list or no lists, browses on one device but purchases on another, uses when not at home and likely price comparing while in stores, responds to discounts, only likes orders with free shipping, etc., etc., etc.—the list is unbounded in length and shopping data experts can extend it greatly. The focus of this disclosure is not the specifics of behaviors that can be tracked. One knowledgeable in any of the fields referenced can easily extend the data sets. The focus of this disclosure is that all behavioral (and genetic and survey and form based) data can be used to determine the likelihood of an individual or group belong not only to an MB Type but to the more granular version of MBTI: MBTI++. Knowing what the typing of an individual or group is can then lead to the next portion of this disclosure which is how that knowledge may be used to make recommendations and optimize user experiences for individuals and groups.

Generating Recommendations Based on MBTI Associated with Each Individual

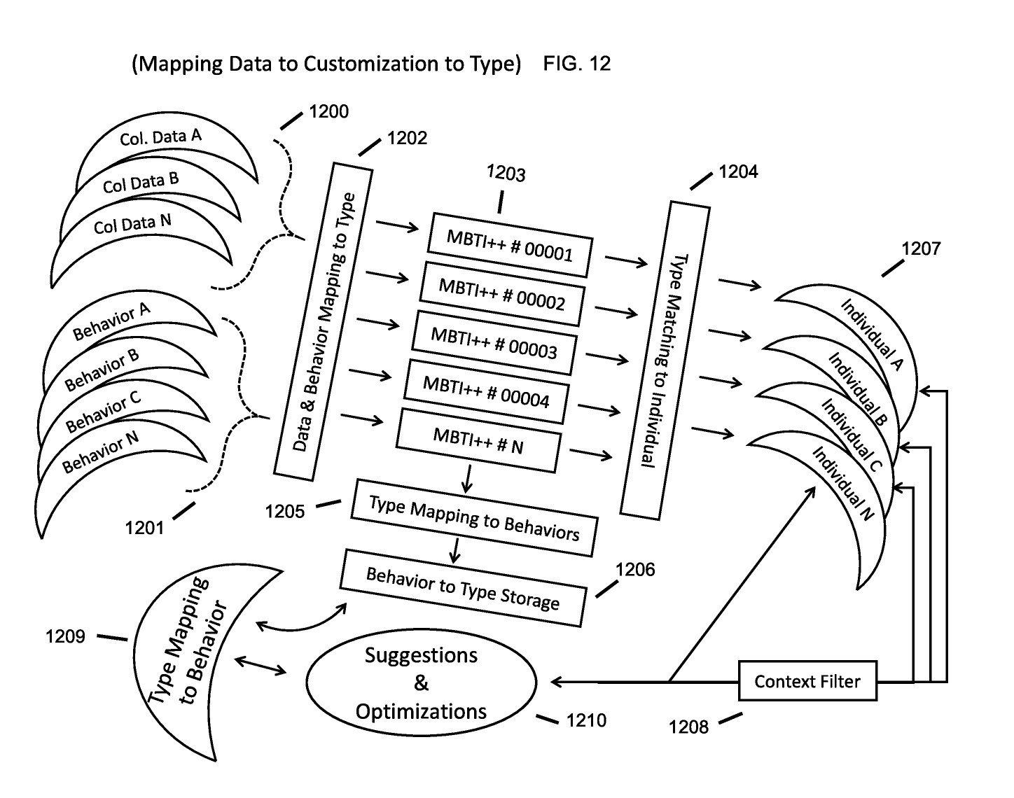

So, now in the process, a reasonable guess may be made about someone's type based on a number of factors including what they (or their genes) say (Collected Data) and what they do (Observable Data). Then, an exercise similar to collaborative filtering may be undertaken, but through the lens of type by looking at the overall data flows but in a slightly different and perhaps more holistic manner than before. To help with this, let's look at FIG. 12. As described earlier in this disclosure, Collected Data (1200) and Behavioral Data (1201) may be mapped (1202) to MBTI+ Types (1203). Now, there may be many—perhaps thousands of MBTI++ types. These types are then matched to Individuals (1204). They are also mapped to behaviors (1205). Though this may seem circular at first—since much of this data was originally collected from the very same users to whom results may be applied—but it is not. The reasoning is as follows: Even ignoring the collected data (which by itself may eliminate the pure circularity) and limiting the analysis to the observed data, the following logic may be applied. There may be 50 different behaviors identified as contributing to the likelihood of one of the MBTI++ Types. These types have all been weighted as to how strongly they represent this particular type. This type is now a weighted set of factors which can be applied not just individually but as a group with each Type weighted when applied to any one individual or group. Suppose for example, I have 2M(illion) people who have contributed their behavior to a belief that their behaviors, more or less contribute to an MBTI++ type, let's call it T12345. Even though an individual may not display the actions that are primarily associated with that type, under different circumstances they might display those actions or, of course, other similar actions that they may have never taken before. A person acting in a certain way at a certain time may be reflective of an MBTI++ Type but other people of that Type may never have taken those actions. Also, actions associated with a specific Type may have never been presented to users who are a particular Type and if presented with those choices, they may not want to take them but that doesn't mean that if they did take them they may not be satisfying. These behaviors (1205) are stored (1206)—likely distributed. This stored data is continuously updated as more data is collected both enhancing the accuracy of existing MBTI++ types and creating new Types.

Now returning to the part of the diagram where the types may be matched to the individuals (1204→1207), there may be one or more likely numerous types that apply to any individual. These types may be weighted (e.g. type ABCDE at a multiplier of 23, type BCDEF at a multiplier of 73, type CDEFG at a multiplier of 17, etc.) but are also contextual. The Context Filter (1208) may mitigate the query based on any factors that could be variable. Some examples (though the potential list is unbounded and could be extremely large) could be: time of day, time of the year, weather, behavior just previous, blood sugar levels, amount of sleep the previous evening, neural activity, alcohol blood level, alcohol blood level 10 hours previous, recent life changes (a loss, a new marriage, a big purchase, a new job, etc.) and, many many more.

Now, on the two branches of the journey, there may be two data sets: 1) MBTI++ type for each individual filtered by context (1207→4208) and 2) Behavior to Type Storage (1205→4206), which feeds the engine which maps type to behavior (1209). In order to make the desired suggestions and optimizations, the two inputs may be provided—the Context filtered typing data (1207/1208) and the behaviors associated with the various types (1209) and compared, scoring the relevance of each behavior to the task at hand. Suggestions and Optimizations Engine (1210) may be used to make suggestions and propose optimizations to the user or more likely the software that is leading or making choices for the user.

Enhancing MBTI++ Using Analysis of Actual Usage

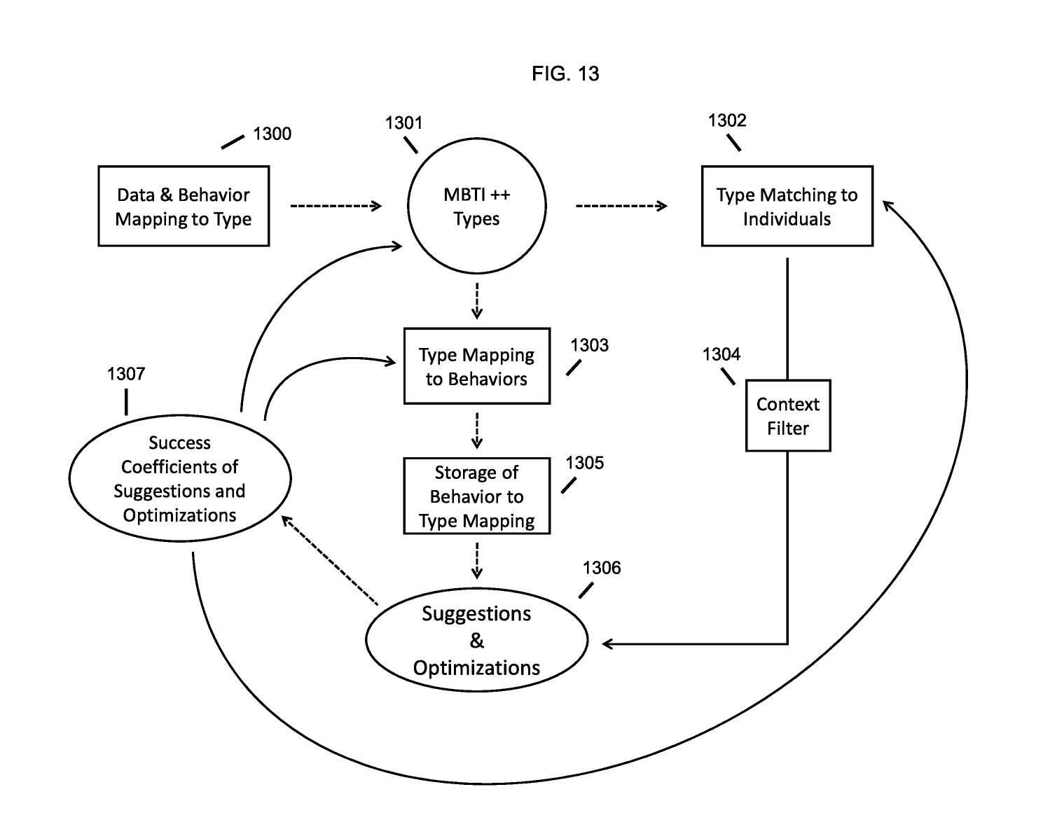

As described above, this system this system is not only iterative but also self-learning. One of the key elements of the system is the feedback loops which are constantly refining their elements based on the success of previous suggestions. As can be seen in FIG. 13, the method may begin as before with Data Mapping and Behavior to Type (1300) from which the MBTI++ Types (1301) may be created. Next, those Types may be mapped to Individuals (1302) and to Behaviors (1303). The Individual Types are filtered by Context (1304) and those filtered Types along with the Behavior to Type Mapping Data (1305) are provided to the Suggestions and Optimizations Engine (1306). This Engine passes all of the data (MBTI++ Types, Mapping to Individuals, Mapping to Behaviors, Filtering by Context and Behavior to Type Mapping Data) is passed to the “Success Coefficients of Suggestions and Optimizations” Engine which analyzes the success of the Suggestions and Optimizations and feeds the relevant data back to the MBTI++ Types, Mapping to Individuals, Mapping to Behaviors. Because of the way various AI engines work, this iterative feedback loop provides a learning mechanism where the AI grades its own performance based on the metrics of success. As in one previous example, type may be used to optimize the shopping cart checkout process, which may indicate that one particular user/behavior analysis increases the completion rate (which is a more optimized behavior analysis). Multiple comparative analyses (e.g., on the order of thousands or even millions of micro analyses) may be performed.

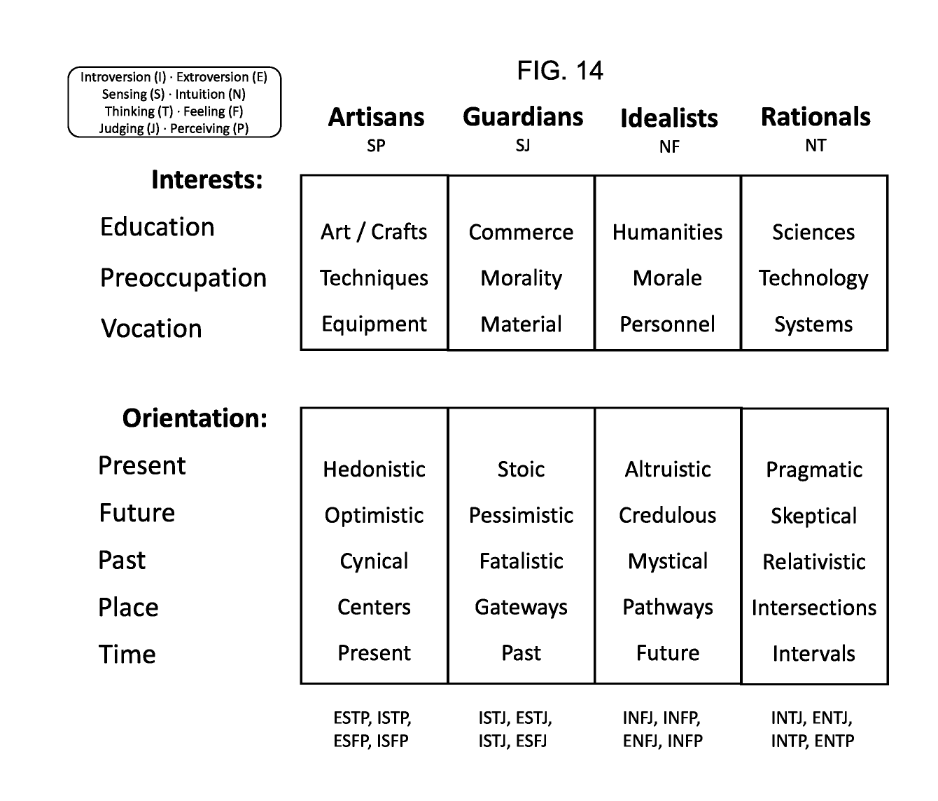

FIG. 14 is a view of predicted behavioral tendencies by personality type. As illustrated, certain personality types may be predicted to have certain behavioral tendencies at high level. The ESTP/ISTP/ESFP/ISFP types, for example, may be predicted to have “Artisan” qualities with interests in art/crafts, preoccupations with technique, vocations dealing with equipment, as well as hedonistic orientations towards the present, optimistic orientations towards the future, cynical orientations towards the past, as well being comfortable in centered places and dealing with the present. Other personality types may be similarly predicted with similar degrees of granularity. As discussed herein, however, such tendencies may be further broken down into even more nuanced categories within each group based on observed behaviors as weighted and refined for each personality category and sub-category.

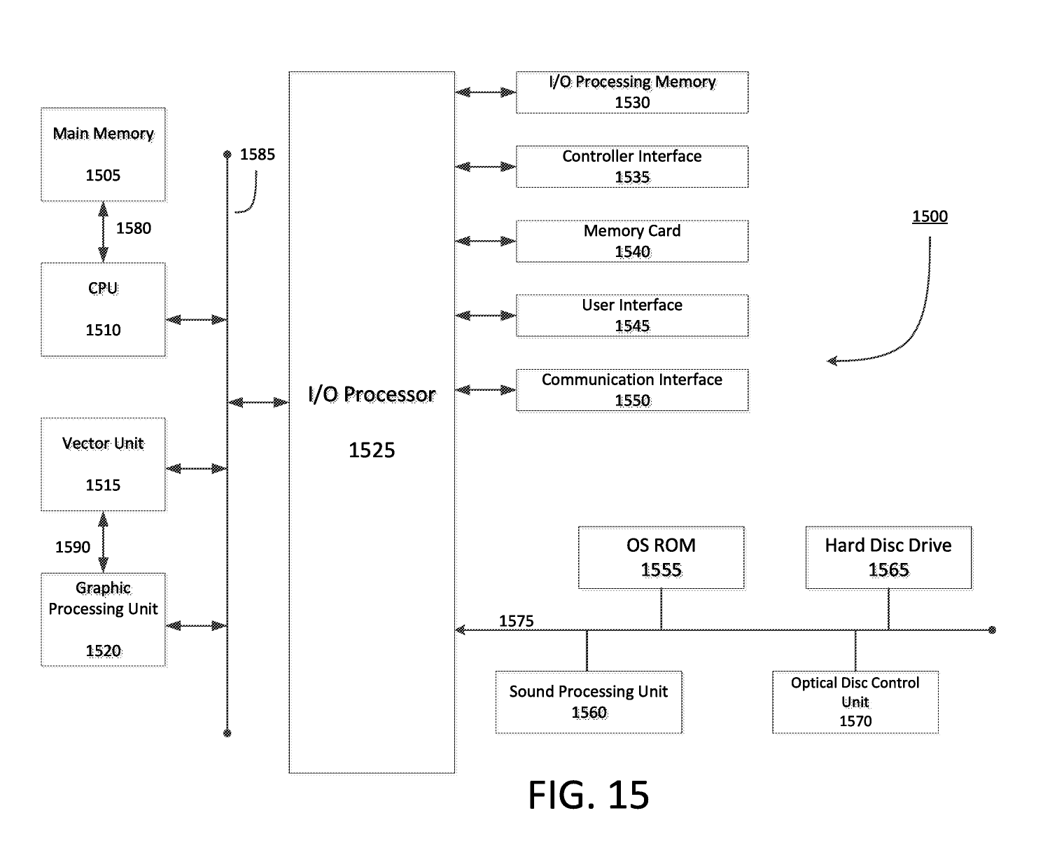

FIG. 15 is a block diagram of an exemplary electronic entertainment system 1500. The entertainment system 1500 of FIG. 15 includes a main memory 1505, a central processing unit (CPU) 1510, vector unit 1515, a graphics processing unit 1520, an input/output (I/O) processor 1525, an I/O processor memory 1530, a controller interface 1535, a memory card 1540, a Universal Serial Bus (USB) interface 1545, and a communication interface 1550. The entertainment system 1500 further includes an operating system read-only memory (OS ROM) 1555, a sound processing unit 1560, an optical disc control unit 1570, and a hard disc drive 1565, which are connected via a bus 1575 to the I/O processor 1525.

Entertainment system 1500 may be an electronic game console. Alternatively, the entertainment system 1500 may be implemented as a general-purpose computer, a set-top box, a hand-held game device, a tablet computing device, or a mobile computing device or phone. Entertainment systems may contain more or less operating components depending on a particular form factor, purpose, or design.

The CPU 1510, the vector unit 1515, the graphics processing unit 1520, and the I/O processor 1525 of FIG. 15 communicate via a system bus 1585. Further, the CPU 1510 of FIG. 15 communicates with the main memory 1505 via a dedicated bus 1580, while the vector unit 1515 and the graphics processing unit 1520 may communicate through a dedicated bus 1590. The CPU 1510 of FIG. 15 executes programs stored in the OS ROM 1555 and the main memory 1505. The main memory 1505 of FIG. 15 may contain pre-stored programs and programs transferred through the I/O Processor 1525 from a CD-ROM, DVD-ROM, or other optical disc (not shown) using the optical disc control unit 1570. I/O Processor 1525 of FIG. 15 may also allow for the introduction of content transferred over a wireless or other communications network (e.g., 4$, LTE, 3G, and so forth). The I/O processor 1525 of FIG. 15 primarily controls data exchanges between the various devices of the entertainment system 1500 including the CPU 1510, the vector unit 1515, the graphics processing unit 1520, and the controller interface 1535.

The graphics processing unit 1520 of FIG. 15 executes graphics instructions received from the CPU 1510 and the vector unit 1515 to produce images for display on a display device (not shown). For example, the vector unit 1515 of FIG. 15 may transform objects from three-dimensional coordinates to two-dimensional coordinates, and send the two-dimensional coordinates to the graphics processing unit 1520. Furthermore, the sound processing unit 1560 executes instructions to produce sound signals that are outputted to an audio device such as speakers (not shown). Other devices may be connected to the entertainment system 1500 via the USB interface 1545, and the communication interface 1550 such as wireless transceivers, which may also be embedded in the system 1500 or as a part of some other component such as a processor.

A user of the entertainment system 1500 of FIG. 15 provides instructions via the controller interface 1535 to the CPU 1510. For example, the user may instruct the CPU 1510 to store certain game information on the memory card 1540 or other non-transitory computer-readable storage media or instruct a character in a game to perform some specified action.

The foregoing description has been directed to specific embodiments. It may be apparent, however, that other variations and modifications may be made to the described embodiments, with the attainment of some or all of their advantages. For instance, it is expressly contemplated that the components and/or elements described herein can be implemented as software being stored on a tangible (non-transitory) computer-readable medium, devices, and memories (e.g., disks/CDs/RAM/EEPROM/etc.) having program instructions executing on a computer, hardware, firmware, or a combination thereof. Further, methods describing the various functions and techniques described herein can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on. In addition, devices implementing methods according to these disclosures can comprise hardware, firmware and/or software, and can take any of a variety of form factors. Typical examples of such form factors include laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example. Instructions, media for conveying such instructions, computing resources for executing them, and other structures for supporting such computing resources are means for providing the functions described in these disclosures. Accordingly this description is to be taken only by way of example and not to otherwise limit the scope of the embodiments herein. Therefore, it is the object of the appended claims to cover all such variations and modifications as come within the true spirit and scope of the embodiments herein

What is claimed is:

1. A system for identifying and targeting personality types and behaviors, the system comprising:
a memory that stores information regarding a subject, the stored information for the subject including a plurality of personality categories, each category associated with a starting percentage;
a communication interface that receives behavioral information from a plurality of sources regarding the subject, wherein the received behavioral information associates one of the personality categories with an observed behavior by the subject; and
a processor that executes instructions stored in memory, wherein execution of the instructions by the processor:
analyzes the behavioral information, wherein analyzing the behavioral information includes identifying a weight for the observed behavior based on the associated personality category,
updates the stored information regarding the subject based on the starting percentage of the observed behavior as weighted by the identified weight; and
generates a report based on an analysis of the updated stored information, the report including a plurality of personality axes, each axis reflecting a weighted percentage for an associated personality category.

2. The system of claim 1, wherein the received behavioral information includes information regarding Internet usage by the subject.

3. The system of claim 1, wherein the received behavioral information includes information regarding commercial activity by the subject.

4. The system of claim 1, wherein the received behavioral information includes information regarding professional activity by the subject.

5. The system of claim 1, wherein the report further includes a recommendation for optimizing a user experience for the subject.

6. The system of claim 5, wherein the recommendation is based on filtering a plurality of user experience options using at least one associated personality category of the subject.

7. The system of claim 5, wherein the communication interface further receives information regarding the optimized user experience that includes an observed interaction by the subject.

8. The system of claim 7, wherein the processor further executes instructions stored in memory to adjust the user experience based on the observed interaction by the subject.

9. The system of claim 7, wherein the memory is further updated regarding the observed interaction by the subject, and wherein the processor generates a report for another subject having a common weighted percentage for a common personality category based on the observed interaction by the subject.

10. A method for identifying and targeting personality types and behaviors, the method comprising:
storing information in memory regarding a subject, the stored information for the subject including a plurality of personality categories, each category associated with a starting percentage;
receiving behavioral information via a communication interface from a plurality of sources regarding the subject, wherein the received behavioral information associates one of the personality categories with an observed behavior by the subject; and
executing instructions stored in memory, wherein execution of the instructions by a processor:
analyzes the behavioral information, wherein analyzing the behavioral information includes identifying a weight for the observed behavior based on the associated personality category,
updates the stored information regarding the subject based on the starting percentage of the observed behavior as weighted by the identified weight; and
generates a report based on an analysis of the updated stored information, the report including a plurality of personality axes, each axis reflecting a weighted percentage for an associated personality category.

11. The method of claim 10, wherein the received behavioral information includes information regarding Internet usage by the subject.

12. The method of claim 10, wherein the received behavioral information includes information behavioral information regarding commercial activity by the subject.

13. The method of claim 10, wherein the received behavioral information includes information regarding professional activity by the subject.

14. The method of claim 10, wherein the report further includes a recommendation for optimizing a user experience for the subject.

15. The method of claim 14, wherein the recommendation is based on filtering a plurality of user experience options using at least one associated personality category of the subject.

16. The method of claim 14, further comprising receiving information regarding the optimized user experience that includes an observed interaction by the subject.

17. The method of claim 16, further comprising executing instructions stored in memory to adjust the user experience based on the observed interaction by the subject.

18. The method of claim 16, further comprising updating the memory regarding the observed interaction by the subject, wherein generating a report for another subject having a common weighted percentage for a common personality category based on the observed interaction by the subject.

19. A non-transitory computer-readable storage medium, having embodied thereon a program executable by a processor to perform a method for identifying and targeting personality types and behaviors, the method comprising:
storing information regarding a subject, the stored information for the subject including a plurality of personality categories, each category associated with a starting percentage;
receiving behavioral information from a plurality of sources regarding the subject, wherein the received behavioral information associates one of the personality categories with an observed behavior by the subject;
analyzing the behavioral information, wherein analyzing the behavioral information includes identifying a weight for the observed behavior based on the associated personality category;
updating the stored information regarding the subject based on the starting percentage of the observed behavior as weighted by the identified weight; and
generating a report based on an analysis of the updated stored information, the report including a plurality of personality axes, each axis reflecting a weighted percentage for an associated personality category.
